(19) United States
(12) Patent Application Publication
(10) Pub. No.: US 2011/0137148 A1
(43) Pub. Date: Jun. 9, 2011

(54) Method and device comprising fused ultrasound and magnetic resonance imaging

(75) Inventor: Vitaliy Khizhnichenko, Irvine, CA (US)

(73) Assignee: Irvine Sensors Corporation, Costa Mesa, CA (US)

(21) Appl. No.: 12/927,499

(22) Filed: Nov. 15, 2010

Related U.S. Application Data
(60) Provisional application No. 61/283,566, filed on Dec. 7, 2009.

(51) Int. Cl. A61B 5/055

(52) U.S. Cl. 600/411

(57) ABSTRACT

A device and method for concurrently obtaining ultrasound and MRI image data are disclosed. A probe member comprises ultrasound imaging means at a first predetermined position on the probe member and an MRI coil at a second predetermined position on the probe member. An MRI imaging system having an MRI pulse sequence comprising an MRI image data acquisition period and an MRI pulse sequence time gap generates an MRI image data set. An ultrasound image data set is acquired only during the MRI pulse sequence time gap in which the operation of the ultrasound imaging means does not interfere with the MRI imaging operation.

### CROSS-REFERENCE TO RELATED APPLICATIONS

This application claims the benefit of U.S. Provisional Patent Application No. 61/283,566, filed on Dec. 7, 2009 entitled “Method for Prostate Cancer Detection Enhancement by Fusing MRI and US Imagery” pursuant to 35 USC 119, which application is incorporated fully herein by reference.

### STATEMENT REGARDING FEDERALLY SPONSORED RESEARCH AND DEVELOPMENT

N/A

### BACKGROUND OF THE INVENTION

1. Field of the Invention

The invention generally relates to the field of magnetic resonance imaging (also referred to as “MRI” herein) and ultrasound imaging.

More specifically, the invention relates to a method and device for applications such as medical imaging of a human subject of a region, (e.g., prostate or uterine imaging) using a probe member. The method and device provide richer content and enhanced imaging by the fusing of an ultrasound imaging modality within and during the MRI pulse sequence of an MRI modality resulting in the concurrent acquisition of digitized image data from both the MRI and ultrasound modalities for subsequent image processing such as fused image registration of the two modalities.

2. Description of the Related Art

Prostate cancer is the most common non-skin cancer in the USA, affecting one in six men. According to the National Cancer Institute, estimated new cases of prostate cancer in the Untied States in 2008 totaled about 186,320 and the estimated number of deaths from prostate cancer totaled about 28,660. It is well-accepted that early diagnosis of prostate, cervical or uterine cancer is crucial in their treatment and a number of medical imaging modalities are important in the early identification and diagnosis of prostate cancer as well as other cancers and medical conditions.

The various imaging modalities used in cancer and lesion identification provide specific information (or descriptors) about a form of cancer or lesion. Fusion of two or more modalities desirably augments the probability of early lesion detection; particularly, prostate cancer.

Image co-registration software is currently used to fuse image data acquired from different image modalities, resulting in the requirement that every pixel of a fused image be a vector with different descriptors as components. Desirably, fused imagery is obtained when component images from different imaging modalities are obtained concurrently.

Integrated (hybrid) imaging systems exist such as GE Discovery ST, Philips GEMINI TF or Siemens Biograph TruePoint PET/CT, which are capable of providing fused, three-dimensional (3-D) PET/CT (i.e., Positron Emission Tomography and Computed Tomography, respectively) concurrently acquired images of an entire human subject including the prostate area. These same companies produce SPECT/CT (SPECT stands for Single Photon Emission Computed Tomography) scanners, capable of providing fused 3-D images with different combination of properties from the above hybrid systems. Unfortunately, these modalities all produce varying levels of harmful ionizing radiation.

Prior attempts have been made to fuse PET and MRI imagery by using two scanners and moving a table with the human subject on it between the scanners. These prior art methods still have the disadvantage of non-concurrence of the two imaging events and resultant likelihood of changes in the subject's body position and lack of image pixel registration between the different images during the imaging event.

All the above image data fusing systems are generally intended to scan the whole human body or a considerable portion or region of a human body and are not tailored specially to small defined regions such as the prostate or cervix. Further, the above PET/CT, SPECT/CT or PET/MRI combinations of modalities (i.e. the corresponding set of descriptors) may be not the most appropriate or useful for prostate lesion detection. For instance, the resolution and signal-to-noise ratio of the above-referenced modalities are frequently insufficient to detect relatively small initial cancer lesions, when diagnosis and treatment are most likely to be successful.

Other suitable imaging modalities include ultrasound and magnetic resonance imaging which are common medical imaging modalities and provide additional beneficial descriptors of for instance, prostate lesion detection.

Literature suggests the MRI and ultrasound combination provides the highest rate of prostate cancer detection. (W. E. Brant, C. A. Helms; Fundamentals of Diagnostic Radiology, 2nd Edition, Lippincott Williams & Wilkins; p. 1460 (1999)). A significant advantage of both MRI and ultrasound as compared to the above-referenced CT, PET and SPECT modalities is that MRI and ultrasound don't ionize living tissue and are therefore harmless for human body.

Experiments have been conducted to prove the feasibility of concurrent MRI/ultrasound imaging as applied to breast biopsy. (A. M. Tang et al., Simultaneous Ultrasound and MRI System for Breast Biopsy: Compatibility Assessment and Demonstration in a Dual Modality Phantom, IEEE Transactions on Medical Imaging, Vol. 27, No. 2, pp. 247-254 (2008)).

In the case of concurrent MRI and ultrasound imaging data acquisition, a major technical concern is cross-talk between the two modalities. Similarly, interference may pose a problem relative to prostate imaging and other internal imaging procedures.

Specifically, endorectal ultrasound transducers in an ultrasound imaging transceiver system typically function in the 7-10 MHz band and therefore the electric signal feeding the ultrasound transducer's piezo-elements is approximately the same order of magnitude.

Of related concern, the gyro-magnetic ratio for hydrogen is about 42.58 MHz/T and the magnetic fields available for MRI imaging systems are usually in the range from about 0.02 to about 4 T. In other words, the MRI radio receiver equipment will pick up signals in a wide range of carrying frequencies between 0.85 and 170.32 MHz. As a result, the concurrent acquisition of MRI and ultrasound image data where the MRI coil and ultrasound transceiver are in close proximity will cause the operation of the ultrasound components to potentially interfere with MRI and special measures must be taken to permit ultrasound and MRI to function simultaneously.

A prior art approach to achieve electromagnetic compatibility between ultrasound and MRI components is the careful isolation of the wires supporting the ultrasound components. Unfortunately, in the case of endorectal devices, this is difficult in that the piezo-electric elements of a transducer in an ultrasound transceiver are generally protected by little more than protective shell fabricated from a plastic or elastomeric material.

Despite the above deficiencies and obstacles, MRI and ultrasound imaging modalities each compensate for the weaknesses of the other when efficiently integrated and fusion of the two concurrently acquired image modalities is desirable.

In particular, MRI has excellent soft tissue contrast but a relatively long imaging time. Further, MRI requires specialized and costly facilities.

On the other hand, ultrasound has high temporal resolution, is relatively inexpensive and portable but has relatively low tissue discrimination ability.

The invention takes advantage of these characteristics and enables the integration or fusing of ultrasound imaging with MRI with minimum MRI facility modification or impact and provides a method for fusing ultrasonic and MRI modalities to permit the concurrent acquisition of image data from both modalities and which permits the above image fusing in a compact device for use in, for instance, endorectal instrumentation.

### BRIEF SUMMARY OF THE INVENTION

A method for generating a fused ultrasound and MRI image of a region such as a prostate in a human subject is disclosed.

In a first aspect, the method of the invention comprises providing a probe member comprising ultrasound imaging means such as an ultrasound transceiver. The probe member is positioned within the subject a predetermined distance from the region to be imaged and the subject and probe member positioned within an MRI system,

The region is imaged with the MRI system to define an MRI image data set where the MRI system utilizes a predetermined MRI pulse sequence comprising a repetition period or TR period. The TR period comprises an MRI image data acquisition period also referred to as a signal readout period followed by an MRI pulse sequence time gap which is the inactive period in the MRI imaging pulse sequence between the end of the MRI signal readout period and the beginning of the subsequent MRI pulse sequence.

The region is concurrently imaged with the ultrasound transceiver while the subject is in the MRI system by initiating an ultrasound imaging event during the MRI pulse sequence time gap to define an ultrasound image data set.

In a second aspect of the invention the above method further comprises the step of co-registering the MRI data set and ultrasound image data set to provide a fused image data set.

In a third aspect of the invention the MRI system further comprises an endorectal MRI coil disposed on the probe member and proximal the ultrasound transceiver on the probe member.

In a fourth aspect of the invention, the MRI pulse sequence comprises a spin echo MRI pulse sequence.

In a fifth aspect of the invention, an apparatus is disclosed for imaging a region such as the prostate of a human subject comprising an ultrasound system comprising a probe member having an ultrasound transceiver integrated therewith. The apparatus further comprises an MRI system utilizing a predetermined MRI pulse sequence, comprising a TR period having an MRI image data acquisition period followed by an MRI pulse sequence time gap configured to generate a digitized MRI image data set of the region. The ultrasound system is configured to concurrently image the region within the MRI pulse sequence time gap to define an ultrasound image data set.

In a sixth aspect of the invention, the apparatus further comprises co-registration means for co-registering the MRI data set and the ultrasound image data set to provide a fused image data set.

In a seventh aspect of the invention, the MRI system further comprises an endorectal MRI coil disposed on the probe member.

In an eighth aspect of the invention the MRI pulse sequence comprises a spin echo MRI pulse sequence.

While the claimed apparatus and method herein has or will be described for the sake of grammatical fluidity with functional explanations, it is to be understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112, are to be accorded full statutory equivalents under 35 USC 112.

### BRIEF DESCRIPTION OF THE SEVERAL VIEWS OF THE DRAWINGS

FIG. 1 depicts an exemplar MRI pulse sequence.

FIG. 2 depicts three prior art embodiments of MRI antenna coils.

FIG. 3 illustrates a prior art ultrasound probe member.

FIG. 4 depicts a preferred embodiment of the MRI/ultrasound probe member of the invention.

FIG. 5 depicts a human subject in an MRI imaging system during an MRU/ultrasound imaging event.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE INVENTION

Turning now to the figures wherein like numerals define like elements among the several views, a method and device for simultaneous MRI and ultrasound image data acquisition is disclosed.

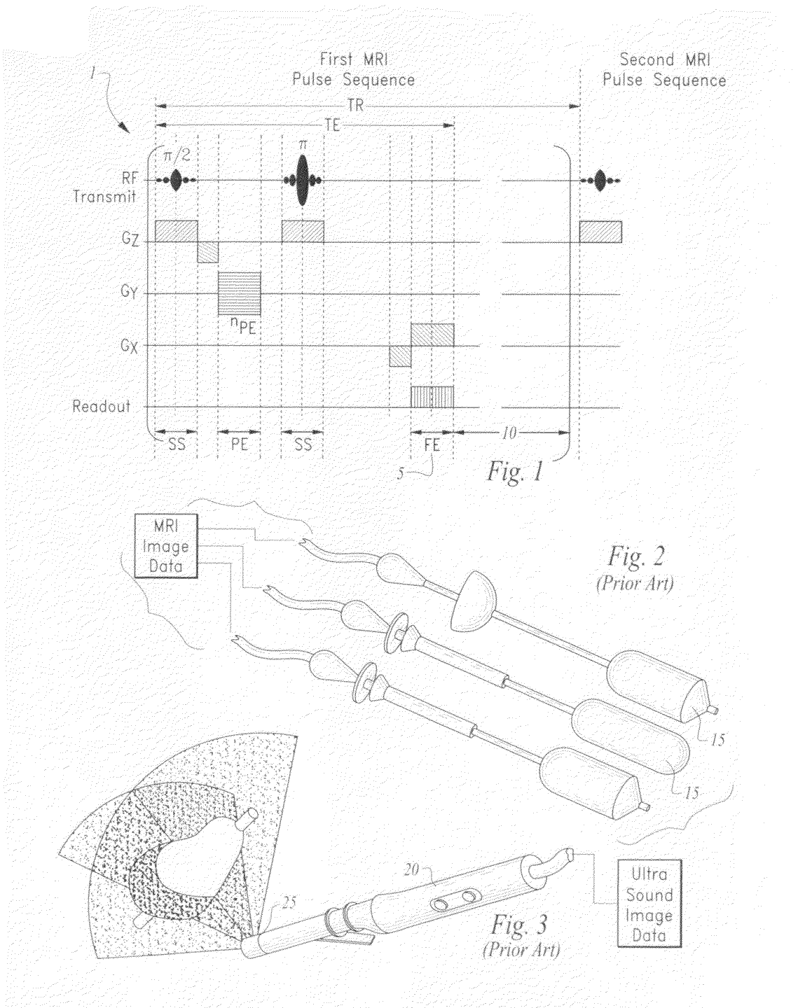

Currently, high quality MRI imaging employs three basic modes of data acquisition: 1) spin density, 2) T1-weighted, and, 3) T2-weighted. Each mode has a different MRI “pulse sequence”. An exemplar MRI pulse sequence 1 is shown in FIG. 1 and comprises at TR period and a TE interval.

A pulse sequence is generally defined as a preselected sequence of defined, precise radio frequency and magnetic gradient pulses that are usually repeated many times during a scan, wherein the time interval between pulses affects the characteristics of the received MR image data. Each MRI pulse sequence has a different “repetition time” (referred to as “TR”) and “echo time” (referred to as “TE”) after which point a read-out gradient is applied.

The TR period comprises an MRI image data acquisition period 5 (also referred to as a read-out period) where the image signal resulting from the RF pulses is received by the MRI receiver, which, in a preferred embodiment, is an MRI receiving antenna coil such as an endorectal coil.

The T1-weighted and T2-weighted modes characterize basic tissue properties and permit the identification of different tissue properties and contrasts. The T1-weighted mode has a time constant based on the rate at which excited protons in the tissue return to equilibrium after RF excitation. The T2-weighted mode is the time constant for loss of phase coherence among spins of protons under the influence of the magnetic gradients applied by the MRI system.

For human tissue, typical time constants for T1 and T2 modes are 0.5 s and 50 ms, respectively.

Table 1 shows characteristic MRI pulse sequence time values TR and TE that are used in the three above MRI modes; i.e., pulse sequence repetition and echo time values for different MRI modes.

| | Spin density | T1-weighted | T2-weighted |
|---|---|---|---|
| TR | 2000-3000 ms | 500-600 ms | 2000-4000 ms |
| TE | ~20 ms | <=20 ms | 80-150 ms |

From Table 1, it can be seen there are relatively significant time gaps (individually referred to as a “MRI pulse sequence time gap” 10 herein) between the end of the MRI image data acquisition period 5 (i.e., the MRI read-out impulse where the returning MRI signal is received by the MRI receiver) and the beginning of the next MRI pulse sequence.

By taking advantage of the MRI pulse sequence time gaps 10 between the ends of each of the MRI image data acquisition periods 5, a fused MRI/ultrasound system is provided for the simultaneous imaging of a region of interest such as the prostate that takes advantage of the beneficial elements of ultrasound and MRI modalities.

FIG. 1 shows an exemplar timing diagram of a typical MRI pulse sequence 1, which, in this figure, Gₓ, G_{y}, G_{z} denote magnetic gradient impulses generated by the MRI system at a predetermined time and intensity in three directions. The letters “SS” stand for “slice selection”, “PE” denotes “phase encoding” and “FE” denotes “frequency encoding”.

The two shaped radio frequency impulses on the first “RF Transmit” line (shown here with a sine modulation) cause a 90° (π/2 radian) nutation of longitudinal nuclear magnetization within a body slice, creating transverse magnetization and a 180° (π radian) rotation of transverse nuclear magnetization within the slice. This transverse magnetization refocuses spins to form a clear spin echo at the MRI data acquisition period (i.e., read-out time).

From FIG. 1 it can be seen that the MRI pulse sequence time gap 10 between the end of the FE impulse and the start of the next “RF transmit” signal is not used by the MRI system and is taken advantage of and employed by the time-gated MRI/ultrasound system of the invention.

As follows from Table 1, this time gap for all of the major MRI modalities can make up more than 90% of the repetition period TR and is used to acquire ultrasound imagery as discussed further below.

Existing commercial MRI systems optionally use positionable MRI receiving coils that act as an MRI antennas, such as the prior art endorectal MRI coils 15 depicted in FIG. 2.

An exemplar prior art transrectal ultrasound probe member 20 comprising ultrasound imaging means 25 is depicted in FIG. 3.

Taking into account that the sound speed in body tissue is equal to approximately c=1540 m/s, with a frequency of 7 MHz (ultrasound transducer frequency), the wavelength is equal to λ=c/f=0.22 mm. Typically, piezo-electric ultrasound sensors in existing ultrasound imaging means are placed with an interval (i.e., a sensor pitch) equal to λ/2=0.11 mm to provide adequate ultrasound beam forming.

An enlarged prostate can reach the size of an orange. Therefore, it can be shown, that for complete coverage of the prostate in the transverse/sagittal plane with a resolution of 0.11 mm, a user can acquire image data frames with sizes 1072×1024 with a frequency of about six fps.

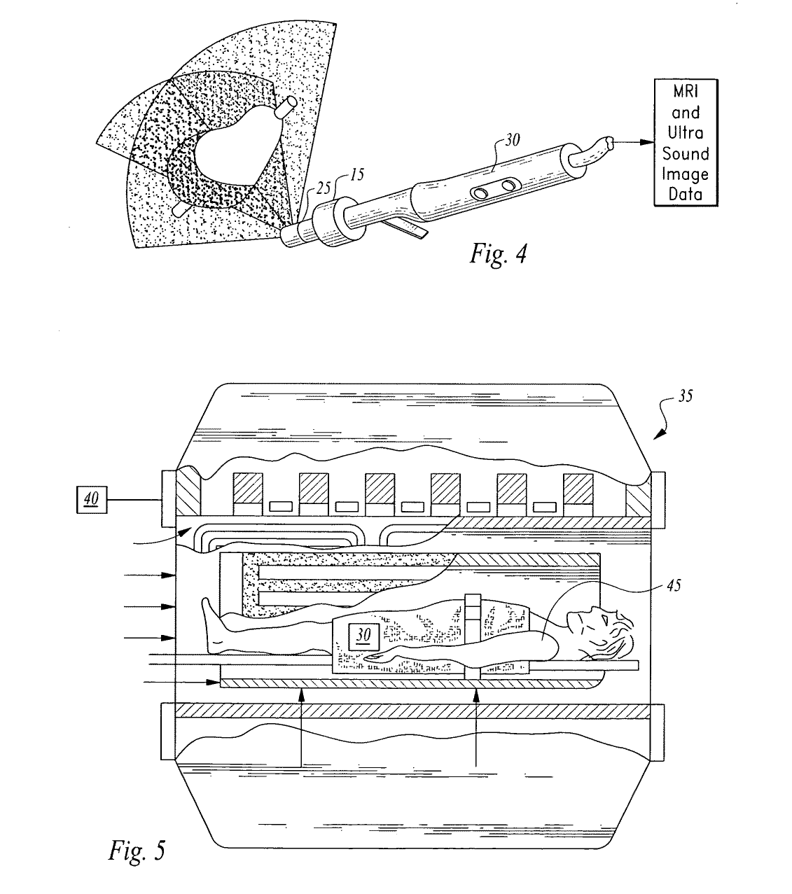

Relating the geometry of the MRI endorectal coils 15 in FIG. 2 with the ultrasound imaging means 25 on ultrasound probe member 20 depicted in FIG. 3, it can be seen these two MRI/ultrasound devices can be combined into one probe member 30 which incorporates ultrasound imaging means 25 and MRI coil 15 by utilizing the method of the invention herein such as depicted in FIG. 4.

Because a voxel (a 3-D picture element) position in an MRI data set is defined only by the relationships between the magnetic field gradients, the MRI receiving coil may be placed anywhere on the probe member of the invention. On the other hand, the ultrasound transducer proximity to the region to be imaged, (e.g., the prostate) is crucial in obtaining high quality imagery.

To minimize the ultrasound system influence on the MRI operation, a time-sharing method is disclosed wherein the ultrasound imaging means 25 such as an ultrasound transceiver or transducer/receiver is active and operational only between in the time gaps between the end of an MRI image data acquisition period 5 and the beginning of the next MRI pulse sequence; typically the first RF excitation pulse and first magnetic gradient pulse in the next MRI pulse sequence of the MRI system.

In a preferred embodiment of the method of the invention, an apparatus for imaging a region of interest is provided comprising a probe member 30 where the probe member comprises ultrasound imaging means 25 such as an ultrasound transceiver or ultrasound transducer and receiver or equivalent ultrasound imaging device at a first predetermined position on the probe member.

An MRI system 35 such as depicted in FIG. 5 is provided utilizing a predetermined MRI pulse sequence wherein the MRI pulse sequence comprises a TR period. The TR period comprises an MRI image data acquisition period 5 followed by an MRI pulse sequence time gap 10 for the generation of an MRI image data set. The MRI system 35 further comprises a positionable MRI receiver antenna coil such as an endorectal MRI coil 15 at a predetermined second position on the probe member 30.

The ultrasound imaging means 25 is configured to image the region of interest during the MRI pulse sequence time gap 10 to produce an ultrasound image data set.

The MRI pulse sequence is initiated by the MRI system 35 to produce an MRI image data set that, in a preferred embodiment, is received by the MRI coil 15 during the MRI image data acquisition period 5.

The MRI pulse sequence then proceeds to the MRI pulse sequence time gap 10 after MRI the signal read out at which point, the ultrasound imaging means 25 is activated to image the region of interest with the ultrasound signal and the ultrasound signal received by the ultrasound imaging means 25 to produce an ultrasound image data set.

As the MRI pulse sequence proceeds to the first step at the beginning of the next MRI pulse sequence, in this instance the generation of an RF pulse, the ultrasound imaging means 25 is terminated a predetermined time beforehand such that the ultrasound imaging means 25 is only transmitting and received during the MRI pulse sequence time gap 10 and is inactive during the remainder of the MRI pulse sequence steps in the MRI imaging process.

Co-registration means 40 such as computer image processing equipment with suitable image processing software is provided for the co-registering the MRI image data set and the ultrasound image data set to provide a fused image data set.

In an alternative preferred embodiment of the method of the invention for imaging a region, a probe member 30 comprising an ultrasound imaging means 25 such as an ultrasound transceiver similar to a prior art transrectal ultrasound probe is provided.

A positionable MRI coil 15 (e.g., an endorectal MRI coil) is disposed on the probe member 30 so as to be proximal the region to be imaged such that MRI image data can be satisfactorily obtained therefrom.

The probe member 30 is positioned (such as transrectally) with respect to the region to be imaged in the subject 45 in similar manner to that used for ultrasound prostate examinations.

The subject and probe member are positioned within an MRI system 35.

The region is imaged with the MRI system 35 to define an MRI image data set wherein the MRI system 35 utilizes a predetermined MRI pulse sequence. The MRI pulse sequence comprises a TR period, which in turn comprises an MRI image data acquisition period followed by an MRI pulse sequence time gap 10.

A typical MRI pulse sequence is a spin echo, T1-weighted or T2-weighted sequence but the method and device of the invention can be implemented using any MRI pulse sequence having a sufficient MRI pulse sequence time gap 10 for the gating of the ultrasound and MRI imaging system operations.

The region is imaged with the ultrasound imaging means 25 during the MRI pulse sequence time gap 10 between the termination of the MRI image data acquisition period 5 of the MRI system 35 and the initiation of the next MRI pulse sequence; here, the first RF impulse of the next MRI pulse sequence. In this manner, a user can define an ultrasound image data set concurrent with MRI operation so as to “time multiplex” between MRI and ultrasound imaging operations without the risk of interference between the operations of the two modalities.

The obtained MRI image data set and ultrasound image data set are uploaded to co-registration means 40 such as computer image processing equipment with suitable image processing software is provided for the co-registration of the respective image data sets to provide an MRI/ultrasound fused image data set.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what essentially incorporates the essential idea of the invention.

I claim:

1. A method for imaging a region comprising:
providing a probe member comprising ultrasound imaging means,
positioning the probe member a predetermined distance from the region to be imaged,
positioning the probe member and region within an MRI system,
imaging the region with the MRI system to define an MRI image data set utilizing a predetermined MRI pulse sequence,
the MRI pulse sequence comprising a TR period comprising an MRI image data acquisition period followed by an MRI pulse sequence time gap,
imaging the region with the ultrasound transceiver within the MRI pulse sequence time gap to define an ultrasound image data set.

2. The method of claim 1 further comprising the step of co-registering the MRI data set and ultrasound image data set to provide a fused image data set.

3. The method of claim 1 wherein the MRI system further comprises a receiving MRI coil disposed on the probe member.

4. The method of claim 1 wherein the MRI pulse sequence comprises a spin echo MRI pulse sequence.

5. The method of claim 3 wherein the MRI system further comprises an endorectal MRI coil disposed on the probe member.

6. The method of claim 3 wherein the MRI pulse sequence comprises a spin echo MRI pulse sequence.

7. An apparatus for imaging a region comprising:
An ultrasound system comprising a probe member comprising an ultrasound transceiver,
an MRI system utilizing a predetermined MRI pulse sequence,
the MRI pulse sequence comprising a TR period comprising an MRI image data acquisition period followed by an MRI pulse sequence time gap to define an MRI image data set,
wherein the ultrasound system is configured to image the region within the MRI pulse sequence time gap to define an ultrasound image data set.

8. The apparatus of claim 7 further comprising co-registration means for co-registering the MRI data set and the ultrasound image data set to provide a fused image data set.

9. The apparatus of claim 7 wherein the MRI system further comprises a receiving MRI coil disposed on the probe member.

10. The apparatus of claim 9 wherein the MRI pulse sequence comprises a spin echo MRI pulse sequence.

11. A probe member comprising ultrasound imaging means at a first predetermined position on the probe member and an MRI coil at a second predetermined position on the probe member.
